(19) United States
(12) Patent Application Publication
(10) Pub. No.: US 2026/0217890 A1
(43) Pub. Date: Jul. 30, 2026

(54) HYBRID POLYMER AND PROCESS FOR PRODUCING SAME

(71) Applicant: SNF GROUP, ANDRÉZIEUX-BOUTHÉON (FR)

(72) Inventors: Céline BESSET, ANDRÉZIEUX-BOUTHÉON (FR); Laura REDELSPERGER, ANDRÉZIEUX-BOUTHÉON (FR); Dimitri MATIOSZEK, ANDRÉZIEUX-BOUTHÉON (FR)

(73) Assignee: SNF GROUP, ANDRÉZIEUX-BOUTHÉON (FR)

(21) Appl. No.: 19/139,996

(22) PCT Filed: Dec. 21, 2023
(86) PCT No.: PCT/EP2023/087447

§ 371 Date: Jun. 17, 2025

(30) Foreign Application Priority Data
Dec. 22, 2022 (FR) FR2214338

(51) Int. Cl. C08F 289/00

(52) U.S. Cl. CPC C08F 289/00

(57) ABSTRACT

A hybrid polymer obtained by a free-radical polymerization process between at least one unsaturated ethylenic monomer and at least one protein. Also, the use of at least one hybrid polymer in a variety of compositions, and the use of compositions including at least one hybrid polymer in numerous fields of application.

### FIELD

The present invention belongs to the technical field of hybrid polymers. More specifically, the subject of the invention is a specific hybrid polymer. Said hybrid polymer is obtained by a free-radical polymerization process between at least one unsaturated ethylenic monomer and at least one protein. The invention also relates to the use of said hybrid polymer in various compositions. The invention also relates to the use of these compositions in various fields of application.

### BACKGROUND

The development of hybrid polymers, i.e. the development of a polymeric entity combining at least two polymers of differing nature, has been the subject of considerable research studies in recent years.

These hybrid polymers have the advantage of exhibiting unique functional properties and application potential, given the multitude of possible polymer combinations, which can lead to new synergies.

These hybrid polymers include hybrid polymers combining biopolymers derived from natural and/or renewable resources and synthetic polymers derived from fossil resources. This type of hybrid polymer is of significant interest in many industries because of their application performance which is similar to synthetic polymers and their sustainable development potential. These characteristics make them attractive polymers in many applications, offering opportunities for the innovation and the development of products that are even more efficient and environmentally friendly.

Many documents present hybrid polymers combining polysaccharides and synthetic polymers or polysaccharides and synthetic monomers. U.S. Pat. Nos. 5,854,191; 5,223,171; 5,227,446 and 5,296,470 disclose the use of such hybrid copolymers in detergency. Documents WO/1810663, WO/18108665 and WO/18108667 themselves disclose such hybrids used in cosmetic formulations.

However, the use of these polysaccharide hybrid polymers remains very specific to given fields of application, mainly in cosmetics. Such polysaccharide hybrids are not found in applications where the hybrid polymers must withstand difficult conditions such as high temperatures or high pressure, such as in petroleum applications or textiles, for example.

It has become more than necessary to develop hybrid polymers, meeting the expectations of various specifications, in various fields of application.

The applicant has thus developed a hybrid polymer combining a monomer unit and protein unit. Said hybrid polymer is included in a composition, which is itself used in various fields of application.

### SUMMARY

One subject of the present invention is a hybrid polymer (HP).

According to a first aspect, the invention relates to a hybrid polymer (HP) comprising monomer units of at least one monomer comprising at least one unsaturated ethylenic function, and protein units, characterized in that the monomer units and the protein units are partially or totally linked by at least one covalent bond, and in that the weight ratio between the monomer units and the protein units is between 50/1 and 1/5.

According to a second aspect, the invention relates to a hybrid polymer (HP) obtained by free-radical polymerization according to a process comprising the following successive steps:
(A) preparing a mixture (M1) comprising at least one monomer comprising at least one unsaturated ethylenic function; and between 0.1% and 50% by weight of at least one protein, optionally at least one solvent;
(B) initiating the polymerization in the mixture (M1) in order to obtain a hybrid polymer (HP).

According to a third aspect, the invention relates to a hybrid polymer (HP) obtained by free-radical inverse emulsion polymerization according to a process comprising the following successive steps:
(A1) preparing an aqueous phase comprising between 1% and 50% by weight of at least one protein, between 4% and 40% by weight of at least one monomer comprising at least one unsaturated ethylenic function and between 20% and 95% by weight of water;
(A2) preparing a lipophilic phase comprising between 70% and 99% by weight of an inert hydrophobic liquid and between 1% and 20% by weight of at least one surfactant;
(A3) mixing said aqueous phase with said lipophilic phase to form an inverse emulsion;
(B) polymerizing said inverse emulsion to obtain an inverse emulsion hybrid polymer (HP).

According to a fourth aspect, the invention relates to a hybrid polymer (HP) obtained by free-radical gel polymerization according to a process comprising the following successive steps:
(A) preparing an aqueous solution comprising between 10% and 40% by weight of at least one monomer comprising at least one unsaturated ethylenic function, and from 1% to 30% by weight of at least one protein, of water, and optionally of at least one pH regulator.
(B) initiating the radical polymerization in the aqueous solution to obtain the hybrid polymer (HP) in the form of a gel;
(C) grinding and drying of the resulting gel to obtain the hybrid polymer (HP) in powder form.

According to a fifth aspect, the invention relates to the use of the hybrid polymer (HP) according to the invention, in particular as a viscosifier.

This aspect of the invention also relates to the use of the hybrid polymer (HP) according to the invention in various compositions, and to the compositions comprising at least one hybrid polymer (HP) according to the invention.

The invention also relates to the use of such compositions in a field chosen from the recovery of hydrocarbons; in the drilling and cementing of wells; in the stimulation of hydrocarbon wells; in the treatment of water; in the treatment of fermentation musts in the treatment of sludges; in paper manufacture; in construction; in the treatment of wood; in the treatment of hydraulic compositions; in the mining industry; in the formulation of cosmetic products; in the formulation of detergents; in textile manufacture; in the manufacture of battery components; in geothermal applications; in the manufacture of diapers; or in agriculture.

### DETAILED DESCRIPTION

### Definitions and Generalities

Throughout the present application, the following definitions apply unless specifically indicated otherwise.

“Hydrophilic monomer” denotes a monomer which has an octanol/water partition coefficient, Kow, of less than or equal to 1, in which the partition coefficient Kow is determined at 25° C. in an octanol/water mixture having a volume ratio of 1/1, at a pH between 6 and 8.

“Hydrophobic monomer” denotes a monomer which has an octanol/water partition coefficient, Kow, of greater than 1, in which the partition coefficient Kow is determined at 25° C. in an octanol/water mixture having a volume ratio of 1/1, at a pH between 6 and 8.

The octanol/water partition coefficient, Kow, represents the ratio of the concentrations (g/l) of a monomer between the octanol phase and the aqueous phase. It is defined as follows:

$$Kow = \frac{[ monomer ] octanol}{[ monomer ] water} \qquad [ Math 1 ]$$

with
[monomer]octanol=equilibrium solubility concentration of the monomer in g/l in n-octanol
[monomer]water=equilibrium concentration of the monomer in g/l in water.

“X and/or Y” is understood according to the invention to mean “X”, or “Y”, or “X and Y”.

All possible combinations of the various embodiments disclosed, whether they are preferred embodiments or embodiments given by way of example, are also part of the invention. In addition, when the ranges of values are indicated, the limits are part of these ranges. The disclosure also includes all combinations between the limits of these ranges of values. For example, the ranges of values “1-20, preferentially 5-15”, means the disclosure of the ranges “1-5”, “1-15”, “5-20” and “15-20” and the values 1, 5, 15 and 20.

According to the present invention, the weight-average molecular weight of the synthetic polymers according to the invention is determined by measuring the intrinsic viscosity. The intrinsic viscosity can be measured by methods known to those skilled in the art and can in particular be calculated from the values of reduced viscosity for various concentrations by a graphical method consisting in plotting the values of reduced viscosity (on the y-axis) as a function of the concentrations (on the x-axis) and extrapolating the curve to zero concentration. The intrinsic viscosity value is read on the y-axis or using the least squares method. Then, the weight-average molecular weight can be determined by the well-known Mark-Houwink equation:

[η]=K·M^{α}
[η] represents the intrinsic viscosity of the polymer determined by the method for measuring the viscosity in solution,
K represents an empirical constant,
M represents the molecular weight of the polymer,
α represents the Mark-Houwink coefficient,
α and K depend on the particular polymer-solvent system. Tables known to those skilled in the art give the values for a and K according to the polymer-solvent system.

The term “monomer unit” denotes the chemical unit relating to the corresponding monomer when it is polymerized in the polymer chain of the polymer.

The term “protein unit” denotes the chemical unit relating to a corresponding protein when it is grafted into the polymer chain of the polymer.

### Hybrid Polymer (HP)

According to a first aspect, the invention relates to a hybrid polymer (HP) comprising monomer units of at least one monomer comprising at least one unsaturated ethylenic function, and protein units, characterized in that the monomer units and the protein units are partially or totally linked by at least one covalent bond, and in that the weight ratio between the monomer units and the protein units is between 50/1 and 1/5.

The weight ratio between the monomer units and the protein units is between 50/1 and 1/5, preferentially between 25/1 and 1/3, more preferentially between 10/1 and 1/2, more preferentially between 6/1 and 1/1.

The hybrid polymer (HP) has a weight-average molecular weight advantageously of between 20 000 and 20 000 000 daltons, preferentially between 100 000 and 15 000 000 daltons.

The hybrid polymer (HP) has a Brookfield viscosity preferentially of between 1000 and 100 000 cPs, more preferentially between 2500 and 75 000 cPs, more preferentially between 5000 and 50 000 cPs. The Brookfield viscosity is measured on a 1% by weight solution of polymer in water with a Brookfield RVT unit viscometer with a rotational speed of 20 rpm.

The hybrid polymer (HP) comprises less than 40% by weight of protein, preferentially from 10% to 50% by weight of protein, more preferentially still from 15% to 45% by weight of protein.

The hybrid polymer (HP) can be in the form of a solution, powder, inverse emulsion, water-in-water dispersion, suspension, or in “clear” form, that is to say in the form of a dispersion of solid polymer particles in an aqueous or oily fluid. When the hybrid polymer (HP) is in powder form, it may be in the form of particles or beads.

According to the invention, the hybrid polymer (HP) can be linear or structured. A structured polymer denotes a non-linear polymer which has side chains so as to obtain, when this polymer is dissolved in water, a high degree of entanglement leading to very high viscosities at low gradient. It is also referred to as a crosslinked polymer.

### Monomers and Monomer Units:

As a reminder, a monomer unit denotes the chemical unit relating to a corresponding monomer when it is polymerized in the hybrid polymer (HP). The constituent monomers of the monomer units of the hybrid polymer (HP) are chosen from monomers comprising at least one unsaturated ethylenic function.

In the present application, the expressions “monomer unit” and monomer are used in an appropriate manner. As regards the hybrid polymer (HP) according to the first aspect of the invention, the expression “monomer unit” is appropriate and used because the monomer reacted during the polymerization and forms part of the polymer chain. As regards the polymer (HP) obtained according to a (free-radical polymerization, or inverse emulsion polymerization, or gel polymerization) process, the expression “monomer” is appropriate and used because the monomer is described in its form before polymerization.

Both expressions can be used in the same sentence, for example a claim, if said claim refers on the one hand to at least one claim of the type “hybrid polymer (HP) comprising . . . ” and on the other hand to at least one claim of the type “hybrid polymer (HP) obtained by a (free-radical polymerization, or inverse emulsion polymerization, or gel polymerization) process”. And the monomers and preferences mentioned below are applicable to both expressions.

Since the monomers may be of synthetic and/or biobased nature, this includes monomers from the following patents: FR3125048, FR3125045, FR3125044, FR3125043, FR3125046, WO2023/281233, EP4175939, WO2023/281088, WO2023/281077, WO2023/281076, WO2023/281078, WO2023/281084, WO2023/281081.

Preferentially, these monomers are hydrophilic monomers chosen from nonionic, anionic, cationic and/or zwitterionic hydrophilic monomers. Preferably, these monomers have a single ethylenic unsaturation (double bond between two carbon atoms).

The nonionic hydrophilic monomers are preferentially chosen from the group comprising water-soluble vinyl monomers, such as acrylamide, methacrylamide, N-alkylacrylamides, N-alkylmethacrylamides, N,N-dialkylacrylamides (for example N,N-dimethylacrylamide or N,N-diethylacrylamide), N,N-dialkylmethacrylamides, alkoxylated esters of acrylic acid, alkoxylated esters of methacrylic acid, N-vinylpyrrolidone, N-methylol (meth)acrylamide, N-vinylcaprolactam, N-vinylformamide (NVF), N-vinylacetamide, N-vinylimidazole, N-vinylsuccinimide, acryloylmorpholine (ACMO), glycidyl methacrylate, glyceryl methacrylate, diacetone acrylamide, methacrylic anhydride, acrylonitrile, maleic anhydride, itaconic anhydride, itaconamide, vinylpyridine, hydroxyalkyl (meth)acrylates, thioalkyl (meth)acrylates, isoprenol and its alkoxylated derivatives, hydroxyethyl (meth)acrylates and their alkoxylated derivatives, hydroxypropyl acrylate and its alkoxylated derivatives, vinyl acetate, and mixtures thereof. Among these nonionic monomers, the alkyl groups are advantageously C1-C5, more advantageously C1-C3, alkyl groups. They are preferentially linear alkyls.

The nonionic monomer units represent between 1 and 99 mol % of the monomer units of the hybrid polymer (HP), preferentially between 2 and 70 mol %.

The anionic hydrophilic monomers are preferentially chosen from acrylic acid; methacrylic acid; dimethylacrylic acid; itaconic acid; crotonic acid; maleic acid; fumaric acid; acrylamidoundecanoic acid; 3-acrylamido-3-methylbutanoic acid; maleic anhydride; monomers of the strong acid type having for example a sulfonic acid or a phosphonic acid type function, such as vinylsulfonic acid, vinylphosphonic acid, allylsulfonic acid, methallylsulfonic acid, 2-methylidenepropane-1,3-disulfonic acid, 2-sulfoethyl methacrylate, sulfopropyl methacrylate, sulfopropyl acrylate, allylphosphonic acid, styrenesulfonic acid, 2-acrylamido-2-methylpropanesulfonic acid (ATBS), 2-acrylamido-2-methylpropanedisulfonic acid; water-soluble salts of these monomers such as their alkali metal salts (different from the crystalline form of the sodium salt of 2-acrylamido-2-methylpropanesulfonic acid), alkaline earth metal salts, or ammonium salts; and mixtures thereof.

The anionic monomer units represent between 1 and 99 mol % of the monomer units of the hybrid polymer (HP), preferentially between 2 and 70 mol %, more preferentially between 3 and 50 mol %.

The term “salified” means the replacement of a proton from at least one acid function of —R(═O)—OH (with R═P, S or C) of the anionic monomer by a metal cation or ammonium cation to form a salt of the —R(═O)—OX type (X being a metal cation or ammonium cation). In other words, the non-salified form corresponds to the acid form of the monomer, for example R—C(═O)—OH in the case of the carboxylic acid function, while the salified form of the monomer corresponds to the form R—C(═O)O⁻X⁺, X⁺ corresponding to a metal cation, preferentially an alkali metal cation, or an ammonium cation. The salification of the acid functions of the polymer can be partial or complete.

The salified form advantageously corresponds to alkali metal (Li, Na, K, etc.) salts, alkaline earth metal (Ca, Mg, etc.) salts or ammonium salts (e.g. ammonium ion or tertiary ammonium salts). The preferred salts are sodium salts.

The salification can be carried out before or after the polymerization.

The cationic hydrophilic monomers are preferentially chosen from monomers derived from units of vinyl type (advantageously acrylamide, acrylic, allyl or maleic type), these monomers possessing a phosphonium function, an ammonium salt or quaternary ammonium salt.

Mention may be made, in particular and in a nonlimiting manner, of diallyldialkylammonium salts such as diallyldimethylammonium chloride (DADMAC); acidified or quaternized salts of dialkylaminoalkylacrylamides; acidified or quaternized salts of dialkylaminoalkylmethacrylamides, such as for example methacrylamidopropyltrimethyl ammonium chloride (MAPTAC), acrylamidopropyltrimethylammonium chloride (APTAC), acidified or quaternized salts of dialkylaminoalkyl acrylate such as quaternized or salified dimethylaminoethyl acrylate (ADAME), acidified or quaternized salts of dialkylaminoalkylmethacrylate, such as quaternized or salified dimethylaminoethyl methacrylate (MADAME), and mixtures thereof. Advantageously, the alkyl groups are C1-C3 alkyl groups.

The cationic monomer units represent between 1 and 99 mol % of the monomer units of the hybrid polymer (HP), preferentially between 2 and 70 mol %, more preferentially between 3 and 60 mol %.

In addition, the present invention also covers monomers of the DADMAC, APTAC and MAPTAC type in which the counterion is a sulfate, a fluoride, bromide or an iodide instead of chloride.

Preferably, the cationic hydrophilic monomer is dimethylaminoethyl methacrylate in its quaternized form.

Those skilled in the art will know how to prepare the quaternized monomers, for example by means of a quaternizing agent of R—X type, R being an alkyl group and X being a halogen or a sulfate.

A “quaternizing agent” denotes a molecule capable of alkylating a tertiary amine.

The quaternizing agent may be chosen from dialkyl sulfates comprising from 1 to 6 carbon atoms or alkyl halides comprising from 1 to 6 carbon atoms. Preferentially, the quaternizing agent is chosen from methyl chloride, benzyl chloride, dimethyl sulfate or diethyl sulfate.

The zwitterionic monomers are preferentially chosen from derivatives having a unit of vinyl type, in particular acrylamide, acrylic, allyl or maleic type. Preferentially, this monomer comprises an amine or quaternary ammonium function and an acid function of carboxylic (or carboxylate), sulfonic (or sulfonate) or phosphoric (or phosphate) type. Mention may be made, in particular and in a non-limiting manner, of derivatives of dimethylaminoethyl acrylate, such as 2-((2-9-(acryloyloxy)ethyl)dimethylammonio) ethane-1-sulfonate, 3-((2-(acryloyloxy)ethyl)dimethylammonio) propane-1-sulfonate, 4-((2-(acryloyloxy)ethyl)dimethylammonio) butane-1-sulfonate, [2-(acryloyloxy)ethyl](dimethylammonio)acetate, derivatives of dimethylaminoethyl methacrylate such as 2-((2-(methacryloyloxy)ethyl)dimethylammonio) ethane-1-sulfonate, 3-((2-(methacryloyloxy)ethyl)dimethylammonio) propane-1-sulfonate, 4-(2-(methacryloyloxy)ethyl)dimethylammonio) butane-1-sulfonate, [2-(methacryloyloxy)ethyl](dimethylammonio)acetate, derivatives of dimethylaminopropylacrylamide such as 2-((3-acrylamidopropyl)dimethylammonio) ethane-1-sulfonate, 3-((3-acrylamidopropyl)dimethylammonio) propane-1-sulfonate, 4-((3-acrylamidopropyl)dimethylammonio) butane-1-sulfonate, [3-(acryloyl)oxy) propyl](dimethylammonio)acetate, dimethylaminopropylmethylacrylamide, 2-((3-methacrylamidopropyl)dimethylammonio) ethane-1-sulfonate, 3-(dimethylammonio) propane-1-sulfonate-4-((3-methacrylamidopropyl)dimethylammonio) butane-1-sulfonate and [3-(methacryloyloxy) propyl](dimethylammonio)acetate, and mixtures thereof. Other zwitterionic monomers are described by the applicant in document WO 21123599.

Preferentially, the preferred hydrophilic monomers are acrylamide, acrylic acid, an oligomer of acrylic acid, 2-acrylamido-2-methylpropanesulfonic acid (ATBS) and/or its salts, N-vinylformamide (NVF), N-vinylpyrrolidone (NVP), dimethyldiallylammonium chloride (DADMAC), quaternized dimethylaminoethyl acrylate (ADAME), quaternized dimethylaminoethyl methacrylate (MADAME).

According to one aspect of the invention, the monomers having an ethylenic function, in addition to being chosen from hydrophilic monomers as described above, can also be chosen from hydrophobic monomers.

Hydrophobic monomers having a Kow partition coefficient greater than 1 can also be used in the preparation of the hybrid polymer (HP). They are advantageously chosen, in particular, from:
esters of (meth)acrylic acid having alkyl, arylalkyl and/or ethoxylated and/or propoxylated chain; derivatives of (meth)acrylamide having alkyl, arylalkyl or dialkyl and/or ethoxylated and/or propoxylated chain; cationic allyl derivatives having alkyl, arylalkyl or dialkyl chain and/or ethoxylated and/or propoxylated chain; hydrophobic anionic or cationic (meth)acryloyl derivatives; and anionic or cationic monomer derivatives of (meth)acrylamide bearing a hydrophobic chain. The alkyl groups of these hydrophobic monomers are preferably C6 to C24 alkyl groups. The most preferred monomers are the bromoalkylated derivatives of methacrylamidodimethylaminopropyl with a C8-C16 alkyl chain and ethoxylated behenyl methacrylate.
n-hexyl (meth)acrylate, n-octyl (meth)acrylate, octyl (meth)acrylamide, lauryl (meth)acrylate, lauryl (meth)acrylamide, myristyl (meth)acrylate, myristyl(meth)acrylamide, pentadecyl (meth)acrylate, pentadecyl(meth)acrylamide, cetyl (meth)acrylate, cetyl(meth)acrylamide, oleyl (meth)acrylate, oleyl(meth)acrylamide, erucyl (meth)acrylate, erucyl(meth)acrylamide, and combinations thereof.
LCST (Lower Critical Solution Temperature) and/or UCST (Upper Critical Solution Temperature) monomers or macromonomers. In other words, the hydrophobic nature may originate from the LCST and/or from the properties of the monomers or macromonomers. Patent application WO2020/094960 A1 refers to such macromonomers having the LCST property.

Among these hydrophobic monomers:
the alkyl groups are preferably C3-C20, more preferentially C3-C8, alkyl groups. The C6-C20 alkyls are preferably linear alkyls whereas the C3-C5 alkyls are preferably branched,
the arylalkyl groups are preferably C7-C25, more preferably C7-C15, arylalkyl groups,
the ethoxylated chains preferably comprise 6 to 100 —CH2-CH2-O— groups, more preferentially 10 to 40,
the propoxylated chains preferably comprise 1 to 50 —CH2-CH2-CH2-O— groups, more preferably 1 to 20.

According to this aspect of the invention, the preferred hydrophobic monomers are chosen from N-isopropylacrylamide; N,N-dimethylacrylamide; N,N-diethylacrylamide; N-tert-butylacrylamide; N-vinylcaprolactam; and diacetone acrylamide.

Still according to this aspect of the invention, the concentration of hydrophobic monomers is preferably between 0.0001 and 10 mol %, more preferentially between 0.001 and 5 mol %, more preferentially still between 0.01 and 3 mol %, more preferentially still between 0.1 and 2 mol %, more preferentially still between 0.2 and 1.5 mol %, more preferentially still between 0.3 and 1.3 mol %, the molar percentage being expressed relative to the total number of moles of monomers in the mixture (M1).

The amounts of the various monomer(s) will be adjusted by those skilled in the art so as not to exceed 100 mol % during the preparation of the water-soluble polymers according to the invention.

### Proteins and Protein Units

As a reminder, a protein unit designates the unit relating to a corresponding protein, when it is grafted into the hybrid polymer (HP).

In the present application, the expressions “protein unit” and “protein” are used in an appropriate manner. As regards the hybrid polymer (HP) according to the first aspect of the invention, the expression “protein unit” is appropriate and used because at least one protein reacted during the polymerization and forms part of the polymer chain. As regards the polymer (HP) obtained according to a (free-radical polymerization, or inverse emulsion polymerization, or gel polymerization) process, the expression “protein” is appropriate and used because the protein is described in its form before polymerization.

Both expressions can be used in the same sentence, for example in a claim, if said claim refers on the one hand to at least one claim of the type “hybrid polymer (HP) comprising . . . ” and on the other hand to at least one claim of the type “hybrid polymer (HP) obtained by a (free-radical polymerization, or inverse emulsion polymerization, or gel polymerization) process”. And the proteins and preferences mentioned below are applicable to both expressions.

The term “protein” is used here to include both native (or chemically unmodified) proteins and modified proteins. The term “modified proteins” means a protein that has undergone one or more pretreatments. These pretreatments can be of physical nature by shearing or of chemical nature by acid or alkaline hydrolysis and/or enzymatic hydrolysis via a protease.

The proteins are obtained by conventional methods known to those skilled in the art, for example by dissolution, milling, screening and classification. The protein(s) can be added in solid or liquid form to the mixture (M1). Advantageously, the proteins are added in powder form.

Advantageously, native proteins are preferred.

The protein(s) constituting the protein unit(s) of the hybrid polymer (HP) may be of animal origin, and examples of animal proteins which may be mentioned include: milk proteins such as β-lactoglobulin, casein and whey; serum proteins such as horse serum; placental proteins; fibrous dermal proteins such as collagen, and elastin, and silk proteins.

The protein(s) according to the invention can be of plant origin, for instance from maize, wheat, barley and oats, soybean or peas, for example, glutelin, prolamin, zein and gluten.

Preferentially, the plant proteins are chosen from soy proteins, wheat proteins, oat proteins, pea proteins. The proteins can be obtained from seeds, for example soybean, cotton seeds, peanuts, sunflower, rapeseed, coconut, flax seeds, sesame, safflower, peas, beans and lentils.

There are also proteins of bacterial and fungal origin, and proteins derived from algae and yeasts.

The proteins may be soluble or insoluble in water. The choice of protein solubility is of real interest in terms of the desired final application.

In order to transform the proteins into a soluble form, it is often necessary to perform digestion by physical, chemical or enzymatic treatment, for example hydrolysis with an acid or alkali, fermentation with yeasts, bacteria or enzymes, extraction methods to remove minor constituents, coagulation from extracts by heating, addition of electrolyte, pH adjustment or addition of precipitants.

Preferentially, the protein(s) selected is (are) soluble in water.

Preferentially, the protein selected will be a casein, a serum protein or wheat protein.

Alternatively, the proteins can be present in the form of a mixture. This means a mixture containing a plurality of proteins originating from the same living kingdom or from different kingdoms. For example, a mixture containing a plurality proteins of animal origin, or a mixture containing at least one protein of animal origin and at least one protein of plant origin and/or one protein of bacterial origin.

The proteins used in the graft copolymerization may be chemically modified in various ways before or after the graft polymerization.

### Hybrid Polymer (HP) Obtained by Free-Radical Polymerization

According to a second aspect, the invention relates to a hybrid polymer (HP) obtained by free-radical polymerization according to a process comprising the following successive steps:
(A) preparing a mixture (M1) comprising at least one monomer comprising at least one unsaturated ethylenic function; and between 0.1% and 50% by weight of at least one protein, optionally at least one solvent;
(B) initiating the polymerization in the mixture (M1) in order to obtain a hybrid polymer (HP).

The mixture (M1) preferentially comprises between 5% and 90% by weight of at least one monomer comprising at least one unsaturated ethylenic function, more preferentially between 10% and 80%, more preferentially between 15% and 80%, more preferentially between 20% and 80%, more preferentially between 25% and 80%, more preferentially between 30% and 80%, more preferentially between 35% and 75%, more preferentially between 40% and 75%, more preferentially between 45% and 75%, more preferentially between 50% and 70%, more preferentially between 55% and 65%.

The mixture (M1) preferentially comprises between 0.1% and 50% by weight of at least one protein, more preferentially between 1% and 40%, more preferentially between 3% and 35%, more preferentially between 5% and 30%, more preferentially between 10% and 25%.

The mixture (M1) preferentially comprises between 1% and 90% by weight of at least one solvent, more preferentially between 5% and 80%, more preferentially between 10% and 70%, more preferentially between 15% and 60%, more preferentially between 20% and 50%.

The invention relates particularly to a hybrid polymer (HP) obtained by a free-radical polymerization process comprising the following successive steps:
(A) preparing a mixture (M1) comprising between 5% and 90% by weight of at least one monomer comprising at least one unsaturated ethylenic function, and between 0.1% and 50% by weight of at least one protein, and at least 1% by weight of a solvent,
(B) initiating the polymerization in the mixture (M1) in order to obtain at least one hybrid polymer (HP).

Suitable solvents can be polar and nonpolar.

Nonpolar solvents include cyclohexane, heptane, benzene, toluene, xylene, ethylbenzene and linear, branched or cyclic alkanes having 2 to 20 carbon atoms, dichloromethane, and ethyl acetate.

Preferentially, the hybrid polymer is polymerized by free-radical polymerization in a polar solvent. The polar solvent then comprises water, an alcohol and/or a ketone. It is possible to use a polar solvent or a mixture of polar solvents.

The alcohol or ketone is preferentially selected from methanol, ethanol, 1-propanol, 2-propanol, 2-methyl-2-propanol, 1 butanol, 2-butanol, dimethyl ketone, diethyl ketone, pentan-2-one, butanone, tetrahydropyran, tetrahydrofuran, 2-methyltetrahydrofuran, 1,3-dioxane, 1,4-dioxane. The mixture of polar solvents is preferably a mixture of protic solvents (proton donors).

The solvent is preferentially water.

The protein(s) are added to the polymerization feedstock, in the mixture (M1) in step (A) of the process described in this second aspect of the present application.

Preferentially, a single protein is added to the polymerization feedstock to form the hybrid polymer (HP).

The polymerization is a free-radical polymerization. Free-radical polymerization includes polymerization using photochemical (UV or radiation) initiators, azo initiators, thermal initiators, or redox salts, and also controlled radical polymerization (CRP) techniques or matrix polymerization techniques.

As controlled radical polymerization techniques, mention may be made, in a non-limiting manner, of techniques such as iodine transfer polymerization (ITP), nitroxide mediated polymerization (NMP), or atom transfer radical polymerization (ATRP), reversible addition-fragmentation chain-transfer (RAFT) polymerization, which includes macromolecular design by interchange of xanthates (MADIX) technology, various variations of polymerizations with organometallic compounds (organometallic mediated radical polymerization (OMRP)), and organoheteroatom-mediated radical polymerization (OHRP).

The polymerization initiators advantageously used may be chosen from compounds which dissociate into radicals under the polymerization conditions, for example: organic peroxides, hydroperoxides, hydrogen peroxide, persulfates, azo compounds and redox catalysts. The use of water-soluble initiators is preferred. In some cases, it is advantageous to use mixtures of various polymerization initiators, for example mixtures of redox salts and azo compounds.

Advantageously, the amount of initiator is between 5 and 1000 ppm, preferentially between 10 and 500 ppm, more preferentially between 20 and 100 ppm, relative to the total weight of the polymerization feedstock.

Said initiator may be added in one go to the mixture (M1), by pouring, or batchwise to the medium.

Preferentially, the polymerization is carried out in a temperature range extending from 20° C. to 160° C., preferably from 30° C. to 100° C. It is preferably carried out at atmospheric pressure.

According to the invention, the hybrid polymer (HP) can be linear or structured. A structured polymer denotes a non-linear polymer which has side chains so as to obtain, when this polymer is dissolved in water, a high degree of entanglement leading to very high viscosities at low gradient. It is also referred to as a crosslinked polymer.

The polymer according to the invention can be structured:
by at least one structural agent, which may be chosen from the group comprising polyethylenically unsaturated monomers (having at least two unsaturated functions), for instance, vinyl, notably allyl, acrylic and epoxy functions, and mention may be made, for example, of methylenebisacrylamide (MBA), triallyamine, or tetraallylammonium chloride or 1,2-dihydroxyethylenebis(N-acrylamide), and/or
by macroinitiators such as polyperoxides, polyazo compounds and polytransfer agents such as polymercaptan (co) polymers, and polyols, and/or
by functionalized polysaccharides.

The amount of branching/crosslinking agent in the monomer mixture is advantageously less than 4% by weight relative to the content (weight) of monomers, more advantageously less than 1%, and even more advantageously less than 0.5%. According to a particular embodiment, it may be at least equal to 0.00001% by weight relative to the content of monomers.

The polymerization is preferably carried out in an inert gas atmosphere in the absence of atmospheric oxygen, for example using nitrogen, argon or carbon dioxide as inert gas, or using enzymatic deoxygenation.

Advantageously, the monomers comprising at least one unsaturated ethylenic function to be polymerized and the protein are introduced into the reaction vessel at the start with at least one polymerization primer and polymerized by heating to the optimum polymerization temperature. It can be advantageous to use two or more proteins. The terms “polymerization initiator” and “polymerization primer” correspond to the same product having the same function, that of starting the polymerization reaction.

The order in which the reactants are metered into the polymerization reactor can be freely modified. If a plurality of monomers are used in the graft copolymerization, the individual monomers can be metered into the polymerization zone successively, or in the form of a mixture or else simultaneously from separate metering means. For example, it is possible to heat a solution or dispersion of the protein in the reactor to the required polymerization temperature and to add the monomers and initiators continuously or in batches.

Similarly, the pH of the reaction medium can exert an influence on the hybrid polymer (HP). The acidic or basic monomers can be used in the form of the corresponding salts. For example, acrylic acid is employed in the form of a free acid or in the form of an alkali metal salt. The polymerization can be carried out in a pH range extending from 1 to 14, preferably from 6 to 12. By modifying the pH it is possible, for example, to precipitate the graft copolymers from solutions. This possibility can be used during the treatment, the purification and the isolation of the graft copolymers.

According to the invention, the hybrid polymer (HP) can have a linear, branched, star-shaped or comb-type branched structure. This structure can be obtained, according to the general knowledge of those skilled in the art, for example by selecting the initiator, the transfer agent, the polymerization technique, such as reversible addition fragmentation chain transfer polymerization (RAFT), nitroxide-mediated polymerization (NMP) or atom transfer radical polymerization (ATRP), the incorporation of structural monomers, or the concentration.

The hybrid polymer (HP) obtained by free-radical polymerization may be in the forms described in the abovementioned “Hybrid polymer (HP)” section. The elements relating to the weight-average molecular weight and the Brookfield viscosity of the polymer which are described above are applicable to the hybrid polymer (HP) obtained according to this aspect of the invention.

### Hybrid Polymer (HP) Obtained by Free-Radical Inverse Emulsion Polymerization

According to a third aspect, the invention relates to a hybrid polymer (HP) obtained by free-radical polymerization, said free-radical polymerization being an inverse emulsion polymerization. The polymer (HP) is then in the form of an inverse emulsion or a powder obtained by drying the inverse emulsion.

In the present application, the expression “inverse emulsion” denotes both inverse emulsions and inverse microemulsions. Inverse emulsion polymerization, that is to say water-in-oil emulsion polymerization, consists in emulsifying an aqueous phase comprising the monomer(s) and the protein, in an organic phase. This emulsification is carried out by means of a water-in-oil inverting agent.

In the present invention, the term “inverting agent” denotes an agent capable of emulsifying oil in water. More specifically, an inverting agent is considered to be a surface-active agent with an HLB greater than or equal to 10, and a surfactant is considered to a surface-active agent with an HLB strictly less than 10. A surface-active agent having an HLB of between 8 and 10 is considered to be a wetting agent. A person skilled in the art may refer to the document “Handbook of applied Surface and Colloid Chemistry” by K. Holmberg, Chapter 11, if necessary.

The hydrophilic-lipophilic balance (HLB) of a chemical compound is a measure of its hydrophilic and/or lipophilic properties, determined by calculating the values for the different regions of the molecule, as described by Griffin in 1949 (Griffin WC, Classification of Surface-Active Agents by HLB, Journal of the Society of Cosmetic Chemists, 1949, 1, pages 311-326).

In the present invention, we adopted the Griffin method based on the calculation of a value based on the chemical groups of the molecule. Griffin assigned a dimensionless number between 0 and 20 to give information on the solubility in water and oil. Substances with an HLB value of 10 are distributed between the two phases so that the hydrophilic group (molecular mass Mh) is projected completely into the water while the hydrophobic group (molecular mass Mp) is adsorbed into the non-aqueous phase.

The HLB value of a substance with a total molecular mass M, a hydrophilic part with a molecular mass Mh and a hydrophobic part with a molecular mass Mp is given by:
HLB=20 (Mh/Mp)

According to this third aspect, the invention relates to a hybrid polymer (HP) obtained by free-radical inverse emulsion polymerization according to a process comprising the following successive steps:
(A1) preparing an aqueous phase comprising between 1% and 50% by weight of at least one protein, between 4% and 40% by weight of at least one monomer comprising at least one unsaturated ethylenic function and between 20% and 95% by weight of water;
(A2) preparing a lipophilic phase comprising between 70% and 99% by weight of an inert hydrophobic liquid and between 1% and 20% by weight of at least one surfactant;
(A3) mixing said aqueous phase with said lipophilic phase to form an inverse emulsion;
(B) polymerizing said inverse emulsion to obtain an inverse emulsion hybrid polymer (HP).

Steps (A1), (A2) and (A3) correspond to sub-steps of step (A) in the free-radical polymerization process for obtaining the hybrid polymer (HP) according to the second aspect of the invention. The inverse emulsion obtained in step (A3) corresponds to the mixture (M1) from step (A) in the free-radical polymerization process for obtaining the hybrid polymer (HP) according to the second aspect of the invention.

The weight ratio of the aqueous phase to the lipophilic phase is preferably from 20/80 to 80/20, more preferentially from 70/30 to 30/70.

An inverting agent is preferentially added to the inverse emulsion after the polymerization step.

In step (A1), the aqueous phase is prepared by mixing at least one protein, at least one hydrophilic, ethylenically unsaturated monomer, and water. The mixture may include other compounds, such as a crosslinking agent.

The aqueous phase of step (A1) comprises from 1% to 50% by weight of at least one protein, preferentially between 1% and 40%, more preferably between by weight. The protein(s) is (are) chosen from those mentioned above, with the same preferences.

The aqueous phase of step (A1) comprises between 4% and 40% by weight of at least one hydrophilic, ethylenically unsaturated monomer, preferably between 10 and 35%, more preferably between 20% and 35% by weight. The monomer(s) comprising at least one ethylenically unsaturated function are chosen from the monomers comprising at least one ethylenically unsaturated function mentioned above with the same preferences.

The aqueous phase of step (A1) comprises between 20% and 95% by weight of water, preferentially between from 25% to 80%, more preferentially between 35% and 75% by weight.

Said aqueous phase is preferentially prepared at a solid concentration of 20% to 45% by weight. Those skilled in the art will know how to adjust the ratios of monomers and protein in accordance with the desired properties.

In step (A2), the lipophilic phase is prepared by mixing at least one inert hydrophobic liquid and at least one surfactant.

The lipophilic phase of step (A2) comprises between 70% and 99% by weight of an inert hydrophobic liquid, preferentially between 75% and 96%, more preferentially between 80% and 93% by weight.

The lipophilic phase also comprises an inert hydrophobic liquid or an inert hydrophobic liquid mixture. The inert hydrophobic liquid advantageously denotes an oil or a water-immiscible solvent. The oil used to prepare the water-in-oil emulsion of the invention may be a mineral oil, a vegetable oil, a synthetic oil or a mixture of several of these oils. Examples of mineral oils are mineral oils containing saturated hydrocarbons of aliphatic, naphthenic, paraffinic, isoparaffinic, cycloparaffinic or naphthyl type. Examples of synthetic oils are hydrogenated polydecene or hydrogenated polyisobutene; an ester such as octyl stearate or butyl oleate. ExxonMobil Exxsol® products are suitable oils.

More specifically, the lipophilic phase consists of a heavy oil, a light oil and a surfactant. Those skilled in the art will know how to adjust the ratios between these three compounds allowing them to obtain a stable system capable of withstanding distillation under reduced pressure.

The lipophilic phase of step (A2) comprises between from 1% to 20% by weight of at least one surfactant, preferentially between 1% and 15%, more preferentially between 3% and 12% by weight.

The lipophilic phase comprises at least one “surfactant”, otherwise referred to as an emulsifier. It corresponds to an agent capable of emulsifying water in oil, more specifically the aqueous phase in the lipophilic phase.

As examples of such a surfactant, mention may be made of surfactant polymers such as polyesters having a molecular weight of between 1000 and 3000, condensation products between a poly(isobutenyl) succinic acid or its anhydride and a polyethylene glycol, block copolymers having a molecular weight of between 2500 and 3500, for example those sold under the names Hypermer®, sorbitan extracts, for example sorbitan monooleate, sorbitan isostearate or sorbitan sesquioleate, polyethoxylated sorbitan esters, or else diethoxylated oleocetyl alcohol or tetraethoxylated lauryl acrylate, condensation products of higher fatty alcohols with ethylene oxide, such as the reaction product of oleyl alcohol with 2 ethylene oxide units; condensation products of alkylphenols and ethylene oxide, such as the reaction product of nonylphenol with 4 ethylene oxide units. Products such as Witcamide® 511, betaine products and ethoxylated amines are also good candidates as emulsifiers.

In a preferred embodiment, the surfactant is sorbitan monoleate, polyethoxylated sorbitan esters or tall oil fatty acid diethanolamine.

The surfactant may also be of biobased nature chosen from sucroesters, alkyl polyglucosides (APGs), diglycerol esters or else phospholipids.

The inverse emulsion may contain at least two surfactants, preferably at least three surfactants, better still at least four surfactants in the lipophilic phase.

The inverse emulsion obtained according to the process preferably contains between 0.8% and 20% by weight of emulsifier, more advantageously 1% to 10% by weight.

In step (A3), the aqueous phase obtained in step (A1) and the lipophilic phase obtained in step (A2) are mixed so as to obtain an inverse emulsion. Generally, a step of emulsification of the two phases is carried out with means known to those skilled in the art.

By way of example, in a reactor equipped with a temperature probe, an inert gas inlet, preferentially for nitrogen, and a mechanical stirrer, the lipophilic phase and then the aqueous phase are added before proceeding with the emulsification and thus obtaining an inverse emulsion, preferentially which is stable.

The medium obtained is then degassed with nitrogen before proceeding with the initiation of the polymerization, the subject of step (B), and thus obtaining the inverse emulsion hybrid polymer (HP). The polymerization is a free-radical polymerization.

In a particular embodiment, once the maximum temperature has been reached, the system is placed under aging.

After polymerization, an inverting agent is added to the inverse emulsion.

The inverting agent is a surface-active agent with an HLB value greater than or equal to 10. As an example of such an inverting agent, reference may be made to ethoxylated sorbitan esters such as sorbitan oleate ethoxylated with 20 equivalents of ethylene oxide (EO 20), sorbitan laurate polyethoxylated with 20 mol of ethylene oxide, castor oil polyethoxylated with 40 mol of ethylene oxide, decaethoxylated oleodecyl alcohol, heptaethoxylated lauryl alcohol or sorbitan monostearate polyethoxylated with 20 mol of ethylene oxide. The inverting agent may also be a polyoxyethylenated alkylphenol; a polyoxyethylenated (10 mol) cetyl ether; a polyoxyethylenated alkylaryl ether; quaternary ammonium derivatives; potassium oleate; N-cetyl-N-ethylmorpholinium ethosulfate; sodium lauryl sulfate; condensation products of higher fatty alcohols with ethylene oxide, such as the reaction product of oleyl alcohol with 10 ethylene oxide units; condensation products of alkylphenols with ethylene oxide, such as the reaction products of isooctylphenol with 12 ethylene oxide units; condensation products of amines of higher fatty acids with five or more ethylene oxide units; tristerylphenol ethylene oxide; ethylene oxide condensation products of partial higher fatty esters of polyhydric alcohols and of internal anhydrides thereof (for example, mannitol anhydride and sorbitol anhydride); amine oxide; an alkyl polyglucoside; a glucamide; a phosphate ester or an alkylbenzenesulfonic acid salt; a water-soluble polymer surfactant.

In a preferred embodiment, the inverting agent is an ethoxylated nonylphenol, preferably having an ethoxylation of 4 to 10, an ethoxy/propoxy alcohol, preferably having a C12 to C25 ethoxy/propoxylation, or an ethoxylated tridecyl alcohol, or an ethoxy/propoxy fatty alcohol.

The inverse emulsion may contain at least two inverting agents, preferably at least three inverting agents, more preferably still at least four inverting agents.

The polymer (HP) obtained by inverse emulsion polymerization is in the form of an inverse emulsion or a powder obtained by drying the inverse emulsion.

It is also possible for the inverse emulsion obtained to be diluted or concentrated. Dilution is generally carried out by adding water and/or oil to the inverse emulsion. It is possible to concentrate the emulsion obtained, for example by distillation. A partially dehydrated inverse emulsion is then obtained.

Generally, distillation under reduced pressure allows the product to be concentrated by removing all or some of the water and the ingredients of the lipophilic phase, such as light oil.

An inverting agent, or a mixture of inverting agents, can be added post-distillation, i.e. at the very end of the process.

According to this third aspect of the invention, the polymer (HP) is in the form of an inverse emulsion. When the polymer (HP), which is in water droplets emulsified in the lipophilic phase, is structured or crosslinked, then the polymer (HP) is in the form of microgels dispersed in the lipophilic phase. This includes hybrid polymers dispersed in the lipophilic main phase. When the inverse emulsion is used in an aqueous phase, the hybrid polymers are then dispersed in the main aqueous phase.

The inverse emulsion polymer (HP) obtained by inverse emulsion polymerization according to the invention preferentially comprises between 10% and 70% by weight of hybrid polymer (HP), relative to the total weight of the inverse emulsion, preferentially between 30% by weight and 60% by weight.

Advantageously, the inverse emulsion comprises at least one hybrid polymer (HP), water (generally between 10% and 50% by weight), an inert hydrophobic liquid (generally between 5% and 50% by weight), at least one surfactant (generally between 1% and 10% by weight), and at least one inverting agent (generally between 1% and 10% by weight). The inverse emulsion polymer (HP) obtained by inverse emulsion polymerization according to the invention can be dried to obtain a powder. One particularly advantageous drying method is spray drying.

In this third aspect according to the invention, the same elements as those mentioned above, including their preferences, are used again, namely: the nature of the polymerization initiators, the amount of said initiators, the nature of the structuring and/or crosslinking agents, the amount of these said structuring and/or crosslinking agents, the molecular weight of the polymer (HP) obtained, the free-radical polymerization, the structure of the hybrid polymer (HP) (linear, structured, etc.).

### Hybrid Polymer (HP) Obtained by Free-Radical Gel Polymerization

According to a fourth aspect of the present invention, the hybrid polymer (HP) is obtained by a free-radical gel polymerization process comprising the following successive steps:
(A) preparing an aqueous solution comprising between 10% and 40% by weight of at least one monomer comprising at least one unsaturated ethylenic function, and from 1% to 30% by weight of at least one protein, water, and optionally at least one pH regulator.
(B) initiating the radical polymerization in the aqueous solution to obtain the hybrid polymer (HP) in the form of a gel;
(C) grinding and drying of the resulting gel to obtain the hybrid polymer (HP) in powder form.

The protein(s) is (are) chosen from those mentioned above, with the same preferences. The monomer(s) comprising at least one ethylenically unsaturated function are chosen from the monomers comprising at least one ethylenically unsaturated function mentioned above with the same preferences.

Before step (B), the aqueous solution is generally degassed, for example with nitrogen to minimize the presence of dioxygen in the aqueous solution. During step (B) one or more polymerization initiators are added. The same polymerization initiators as those mentioned above can be used, in the same amounts.

Steps (A) and (B) are preferentially carried out in the same reactor, called the polymerization reactor.

In a preferred embodiment, the gel obtained in step (B) is aged at a final polymerization temperature of between 80° C. and 150° C. for at least 60 minutes without heating.

According to this aspect, the gel obtained in step (B), and before being ground and dried, comprises between 10% and 70% by weight of hybrid polymer (HP), preferentially between 20% and 60% by weight, even more preferentially between 30% and 55% by weight.

The gel thus obtained is generally transported to a granulator to be cut. The gel thus cut is then dried, ground and screened to obtain the hybrid polymer (HP) in powder form.

The pH regulator is advantageously chosen from the following elements: hydrochloric acid, sulfuric acid, phosphoric acid, nitric acid, citric acid, formic acid, acetic acid, adipic acid, propionic acid, oxalic acid, benzoic acid, sodium hydroxide, potassium hydroxide, sodium carbonate, sodium bicarbonate. Those skilled in the art will know how to define the pH to be reached at the end of step b) and also the amount and choice of pH regulators as a function of the chemistry of the polymer to be synthesized, in particular as a function of the nature of the monomers (cationic, anionic, etc.).

As soon as the polymerization begins, the aqueous solution is heated or heats up (exothermic reaction) depending on the starting conditions chosen. Advantageously, because of the heat of polymerization released, the temperature of the reaction mixture generally rises from 80° C. to 150° C., preferably from 80° C. to 100° C. The polymerization is advantageously carried out at atmospheric pressure.

The reactor used is in most cases jacketed so that the reaction mixture can be cooled or heated as required. Once the polymerization reaction is complete, the gel obtained can be rapidly cooled, for example by cooling the wall of the reactor.

At the end of the polymerization reaction, after left the gel to age for at least 60 minutes, the product resulting from the polymerization is a viscous gel that “is self-supporting”.

Granulation consists in cutting the gel into small pieces. Advantageously, the average size of these pieces of gel is less than 1 cm, more advantageously it is between 4 and 8 mm. Those skilled in the art will know how to choose the means suitable for optimum granulation. Granulation is also described in the section relating to the presentation of the prior art.

The drying means and its conditions (duration+temperature) are routine choices for those skilled in the art. Industrially, drying is advantageously carried out by a fluidized bed or rotor dryer, advantageously using air heated to a temperature between 70° C. and 200° C., the temperature of the air being a function of the nature of the product and of the drying time applied.

On conclusion of the drying, the composition is physically in powder form in order to then be ground and screened.

The grinding step consists in breaking the large polymer particles into smaller particles. This can be done by shearing or by mechanical crushing of the particle between two hard surfaces. Various types of equipment known to those skilled in the art may be used for this purpose. Mention can for example by made of rotor mills, where the particle is crushed on a compression blade by the rotating part, or else roll mills, where the particle is crushed between two rotating cylinders. The screening is then intended to remove, depending on the specifications, particles with a mean size that are too small or too large.

The hybrid polymer (HP) obtained by gel polymerization may be a water-swellable polymer or a superabsorbent. This is the case when the hybrid polymer (HP) is structured or crosslinked with one or more structuring and/or crosslinking agents.

In this fourth aspect according to the invention, the same elements as those mentioned above, including their preferences, are used again, namely: the nature of the polymerization initiators, the amount of said initiators, the nature of the structuring and/or crosslinking agents, the amount of these said structuring and/or crosslinking agents, the molecular weight of the polymer (HP) obtained, the free-radical polymerization, the structure of the hybrid polymer (HP) (linear, structured, etc.).

### Use of the Hybrid Polymer (HP) in Various Applications

In a fifth aspect, the invention relates to the use of the hybrid polymer (HP), in particular as a viscosifier. This aspect of the invention also relates to the use of the hybrid polymer (HP) according to the invention in various compositions, to the use of the hybrid polymer (HP) as viscosifier, to compositions comprising at least one hybrid polymer (HP) according to the invention, and also to the processes using said compositions.

Said use relates to the hybrid polymer (HP) according to the first aspect of the invention or obtained according to the second aspect of the invention (by free-radical polymerization), or obtained according to the third aspect of the invention (by inverse emulsion polymerization), or obtained according to the fourth aspect of the invention (by gel polymerization).

The invention therefore relates to the use of said hybrid polymer (HP) as a viscosifying agent in the recovery of hydrocarbons (petroleum and/or gas); in the drilling and cementing of wells; in the treatment of water in open, closed or semi-closed circuit; in the treatment of fermentation musts; in the treatment of sludges; in paper manufacture; in construction; in the treatment of wood; in the treatment of hydraulic compositions (concrete, cement, mortar and aggregates); in the mining industry; in the formulation of cosmetic products; in the formulation of detergents; in the manufacture of textile printing pastes; in the manufacture of battery components; in geothermal applications; in the manufacture of diapers; or in agriculture.

Advantageously, the invention relates to the use of the hybrid polymer (HP) according to the invention in the manufacture of cosmetic compositions as a thickener (thickening agent), conditioner (conditioning agent), stabilizer (stabilizing agent), emulsifier (emulsifying agent), fixative (fixing agent) or film former (film-forming agent).

The invention also relates to the use of the hybrid polymer (HP) as a viscosifier for a pigment composition used in textile printing, said agent comprising at least one hybrid polymer according to the invention. The invention also relates to the use of the polymer (HP) according to the invention as a superabsorbent.

The hybrid polymer (HP) according to the invention can be used as flocculant, coagulant, binder, fixative, viscosity reducer, thickener, absorbent, friction reducer, dewatering agent, drainage aid, filler retention agent, dehydrating agent, conditioner, stabilizer, film-forming agent, sizing agent, superplasticizer, clay inhibitor or dispersant. The invention also relates to a viscosifier comprising at least one hybrid polymer (HP) according to the invention in a field selected from the recovery of hydrocarbons; in the drilling and cementing of wells; in the stimulation of hydrocarbon wells; in paper manufacture; in construction; in the mining industry; in the formulation of cosmetic products; in the formulation of detergents; in the manufacture of textiles.

Owing to the various possible uses of the present composition, the latter is used in diverse compositions. The invention also relates to an aqueous composition comprising at least one hybrid polymer (HP) according to the invention, preferentially for thickening said composition.

The hybrid polymer (HP) according to the invention is able to thicken aqueous compositions, coagulate, bind, fix, reduce viscosity, have absorbency, reduce friction, dewater, drain, retain, dehydrate, condition, stabilize, fix, form film or size depending on the intended field of application.

The implementation of the composition according to the invention in compositions can be carried out according to the knowledge and practices of the formulators in accordance with the intended field of applications.

Advantageously, the composition according to the invention comprises between 0.001% and 5% by weight of the composition, relative to the total weight of the formulation.

More specifically, use could be made of the composition in a field chosen from the recovery of hydrocarbons; in the drilling and cementing of wells; in the stimulation of hydrocarbon wells; in the treatment of water; in the treatment of fermentation musts; in the treatment of sludges; in paper manufacture; in construction; in the treatment of wood; in the treatment of hydraulic compositions; in the mining industry; in the formulation of cosmetic products; in the formulation of detergents; in textile manufacture; in the manufacture of battery components; in geothermal applications; in the manufacture of diapers; or in agriculture.

The invention also relates to the use of such compositions in a field chosen from the recovery of hydrocarbons; in the drilling and cementing of wells; in the stimulation of hydrocarbon wells; in the treatment of water; in the treatment of fermentation musts; in the treatment of sludges; in paper manufacture; in construction; in the treatment of wood; in the treatment of hydraulic compositions; in the mining industry; in the formulation of cosmetic products; in the formulation of detergents; in textile manufacture; in the manufacture of battery components; in geothermal applications; in the manufacture of diapers; or in agriculture.

The invention also relates to a process for the enhanced recovery of petroleum or gas by flushing a subterranean formation, comprising the use of at least one hybrid polymer (HP) according to the invention.

### EXAMPLES

The examples which follow make it possible to best illustrate the advantages of the invention in a clear and non-limiting manner.

### I—Preparation of Polymers in Inverse Emulsion

### Example 1 (Invention): Synthesis of Hybrid Polymer HP1, in Inverse Emulsion

An aqueous phase is prepared with 24.16% by weight of sodium acrylate, 2.68% by weight of milk serum protein powder, 73.11% by weight of deionized water and 0.04% by weight of Versenex 80, and 0.006% by weight of an azo initiator. The pH is adjusted to 7.0±0.1.

A lipophilic phase is prepared from 40.52% by weight of heavy oil (octyldodecyl myristate), 44.78% by weight of light oil (Isopar J) and the following surfactants: 4.90% by weight of Witcamide®511 (tall oil fatty acid diethanolamine), 1.96% by weight of sorbitan monooleate VG and 7.84% by weight of HYPERMER 6212

The aqueous phase is added to the lipophilic phase while mixing in order to form an emulsion. The resulting dispersion is bubbled with nitrogen for 30 minutes while the temperature is stabilized at 25° C., at which point a 10 ml solution containing 0.002% by weight of tert-butyl hydroperoxide emulsion and a 10 mL solution containing 0.04% by weight of sodium metabisulfite (MBS) emulsion are introduced into the dispersion at a flow rate of 0.1 milliliter per minute. The polymerization temperature is controlled between 38° C. and 42° C. for about 90 minutes. The residual monomers are trapped by introducing a solution containing 0.03% by weight of sodium metabisulfite (MBS) at a flow rate of 1.0 milliliter per minute.

The water-in-oil emulsion is then filtered using a 300 μm filter before being distilled under reduced pressure for 1 h30 at 95° C. between 250 mbar and 80 mbar.

A water-in-oil hybrid polymer emulsion is then obtained.

4.5% by weight of an inverting agent (ethoxylated fatty alcohol: TO 06) is added to the water-in-oil polymer emulsion.

### Example 2 (Invention): Synthesis of Hybrid Polymer HP2, in Inverse Emulsion

An aqueous phase is prepared with 4.21% by weight of acrylamide, 45.79% by weight of an acrylamido 2-(acryloyloxy)ethyl trimethylammonium chloride solution, 5.55% by weight of milk serum protein powder, 44.42% by weight of deionized water, and 0.03% by weight of an azo initiator.

The pH is adjusted to 6.5±0.1.

A lipophilic phase is prepared from 58.95% by weight of heavy oil (octyldodecyl myristate), 28.10% by weight of light oil (Isopar J) and the following surfactants: 4.02% by weight of Witcamide®511 (tall oil fatty acid diethanolamine), 1.79% by weight of sorbitan monooleate VG and 7.14% by weight of HYPERMER 6212

The aqueous phase is added to the lipophilic phase while mixing in order to form an emulsion.

The resulting dispersion is bubbled with nitrogen for 30 minutes while the temperature is stabilized at 25° C., at which point 0.004% by weight of peroxide is added to the emulsion and 10 ml of a solution containing 0.04% by weight of sodium metabisulfite emulsion are introduced into the dispersion at a flow rate of 10 milliliters per hour.

The polymerization temperature is controlled between 38° C. and 42° C. for about 90 minutes.

The water-in-oil emulsion is then filtered using a 300 μm filter before being distilled under reduced pressure for 1 h 30 at 90° C. between 250 mbar and 80 mbar.

A water-in-oil hybrid polymer emulsion is then obtained.

4.5% by weight of a high HLB surface-active agent (TO 06) is added to the water-in-oil polymer emulsion.

### (Counter Example): Synthesis of Polymer P3, in Inverse Emulsion

The same protocol as example 2 was applied, while removing the protein from the formulation.

### Example 3: Viscosity Measurement

The Brookfield viscosity is measured on a 1.5% by weight solution of hybrid polymer in water with a Brookfield RVT unit viscometer with a rotational speed of 20 rpm at 25° C. Table 1 summarizes the results.

TABLE 1
viscosity measurement results

| Polymer | Brookfield RVT viscosity 20 rpm @ 1.5% water (cP) |
|---|---|
| PH₂ | 68 000 cPs |
| P3 | 31 600 cPs |

Hybrid polymer PH₂ develops a viscosity in water at 1.5% that is much higher than that of polymer P3.

In this case, the applicant observes that the integration of the serum protein into this type of emulsion therefore has a real advantage for improving the viscosifying performance of the hybrid polymer according to the invention.

### II—Synthesis of Hybrid Polymer According to the Invention by Gel Process

### Example 4 (Invention): Synthesis of Hybrid Polymer PH₄, by Gel Process

45 g of serum protein are added gradually to an aqueous solution containing 550 g of deionized water, 310 g of acrylamido 2-(acryloyloxy)ethyl trimethylammonium chloride (ADC) and 95 g of acrylamide.

This aqueous solution is placed in a 2 L beaker and then cooled to 0° C. before being placed in a Dewar flask.

This aqueous solution is then homogenized using a hand blender at a speed of 500 rpm for 15 seconds before being degassed under nitrogen bubbling for 15 minutes. The following are then added to the aqueous solution: 0.5 g of azo initiator (V50) and 0.003 g of transfer agent (sodium hypophosphite), then the reaction is initiated by successive additions of 2.6×10⁻⁴ g of tert-butyl hydroperoxide and then 1 g of sodium bisulfite. The reaction time is 60 minutes, for a final temperature of 94° C.

The hybrid polymer PH₄ obtained is in gel form. Therefore, it is possible to granulate it and then dry it in an air flow at 70° C. overnight. The dry grains of polymer PH₄ are then ground.

The polymer PH₄ obtained is 100% water-soluble and has a UL viscosity of 5 g/l=2.96 cPs, LVT unit-60 rpm at 25° C.?

### Example 5 (Invention): Application Testing of PH₄

Flocculation Test on the Sludge of the Andrézieux Wastewater Treatment Plant with a Solids Content of 12.2 g/l:

In order to compare the efficacy of the polymer PH₄, flocculation tests were performed with a reference polymer.

The polymers are tested at a concentration of 3 g/l in deionized water.

Flocculation tests are carried out in a beaker in order to assess the flocculation quality and the amount of water drained after 10 seconds. Table 2 summarizes the results.

TABLE 2
application test results

| | | | Reference polymer | |
|---|---|---|---|---|
| | Polymer PH₄ | | (Acrylamide/ADC 45/55) | |
| Volume (ml) | 4 | 5 | 4 | 5 |
| Dosage (ppm/sludge) | 60 | 75 | 60 | 75 |
| Dosage (kg/T solids) | 4.9 | 6.1 | 4.9 | 6.1 |
| Assessment of the | ++ | ++ | ++ | ++ |
| flocs | | | | |
| Filtrate | Lightly | Lightly | Lightly | Lightly |
| | loaded | loaded | loaded | loaded |

The applicant observes that the polymer PH₄ achieves application performance qualities similar to the reference polymer with a similar dosage.

### Drainage Test:

5 ml of a solution containing 3 g/l of the polymers to be tested (polymer PH₄, reference polymer 1, reference polymer 2) are added to a 400 ml beaker containing 200 ml of sludge. The solutions are then mixed by decanting until flocculation occurs.

A filter cloth is placed in a 90 mm-diameter Buchner funnel, the whole assembly is wetted. The Buchner funnel is placed under a measuring cylinder.

The flocculated sludge solution is poured into the Buchner funnel, and the stopwatch is started.

The volumes discharged after 5 and 10 seconds are recorded.

For a drawn-off volume of 5 ml of a 3 g/l polymer solution. Table 3 summarizes the results.

TABLE 3
Drainage results

| Time (s)/ | | Reference polymer 1 | Reference polymer 2 |
|---|---|---|---|
| volume | Polymer | (Acrylamide/ADC | (Acrylamide/ADC |
| drained (ml) | PH₄ | 45/55) | 45/55) |
| 5 seconds | 94 | 81 | 71 |
| 10 seconds | 115 | 106 | 98 |

Drainage corresponds to the volume of water recovered at a given time; the higher the value, the better the performance of the polymer.

The applicant observes that the polymer PH₄ is a better flocculant than the reference polymers.

### Example 6 (Invention): Synthesis of Hybrid Polymer PH₅, by Gel Process

140 g of serum protein and 10 g of crosslinking agent (methylenebisacrylamide) are added gradually to an aqueous solution containing 670 g of deionized water, 125 g of potassium acrylate and 200 g of acrylamide.

This aqueous solution is placed in a 2 L beaker and then cooled to 0° C. before being placed in a Dewar flask.

This aqueous solution is then homogenized using a hand blender at a speed of 500 rpm for 15 seconds before being degassed under nitrogen bubbling for 10 minutes.

The following are then added to the aqueous solution: 0.21 g of sodium persulfate, 0.15 g of sodium metabisulphite (MBS) and 0.01 g of Mohr's salt. The reaction time is 60 minutes, for a final temperature of 94° C.

The hybrid polymer PH₅ obtained is in gel form, this gel formed is left to age for 2 hours. Therefore, it is possible to granulate it and then dry it in an air flow at 70° C. overnight. The dry grains of polymer PH₅ are then ground.

### Example 7 (Invention): Application Testing of PH₅

### Water-Swellable Application of PH₅

Case 1) 0.5 g of PH₅ powder are added to 750 ml of deionized water.

The mixing is carried out under a fume hood, in a stainless steel rotary drum with a diameter of 20 cm for 3 hours.

Case 2) 0.5 g of PH₅ powder are added to 750 ml of municipal water.

The mixing is carried out under a fume hood, in a stainless steel rotary drum with a diameter of 20 cm for 3 hours.

In each case, the entire mixture obtained is placed on a screen with a 100 μm mesh size, subjected to a vibration with an amplitude of 1.5 mm/“g” (1 g=9.81 m/s2) for 1 minute (Retsch AS 200 Control sieve).

The product retained by the filter is weighed to obtain the absorption rate.

Table 4 summarizes the results.

TABLE 4
retention test results

Retention rate

| | Deionized water | Municipal water |
|---|---|---|
| PH₅ | 430 g/g | 100 g/g |
| Reference | 370 g/g | 125 g/g |

Polymer PH₅ has a similar retention rate to the reference polymer.

1-13. (canceled)

14. A hybrid polymer comprising monomer units of at least one monomer comprising at least one unsaturated ethylenic function, and protein units, wherein the monomer units and the protein units are partially or totally linked by at least one covalent bond, and a weight ratio between the monomer units and the protein units is between 50/1 and 1/5.

15. A hybrid polymer obtained by a free-radical polymerization process comprising the following successive steps:
(A) preparing a mixture comprising at least one monomer comprising at least one unsaturated ethylenic function, and between 0.1% and 50% by weight of at least one protein, optionally at least one solvent,
(B) initiating the polymerization in the mixture in order to obtain at least one hybrid polymer.

16. The hybrid polymer according to claim 15, obtained by free-radical inverse emulsion polymerization according to a process comprising the following successive steps:
(A1) preparing an aqueous phase comprising between 1% and 50% by weight of at least one protein, between 4% and 40% by weight of at least one monomer comprising at least one unsaturated ethylenic function and between 20% and 95% by weight of water;
(A2) preparing a lipophilic phase comprising between 70% and 99% by weight of an inert hydrophobic liquid and between 1% and 20% by weight of at least one surfactant;
(A3) mixing said aqueous phase with said lipophilic phase to form an inverse emulsion; and
(B) polymerizing said inverse emulsion to obtain an inverse emulsion hybrid polymer (HP).

17. The hybrid polymer according to claim 14, wherein the monomer units of at least one monomer comprising at least one unsaturated ethylenic function, or the at least one monomer comprising at least one unsaturated ethylenic function, are hydrophilic, and nonionic and/or anionic and/or cationic.

18. The hybrid polymer according to claim 14, wherein the monomer units of at least one monomer comprising at least one unsaturated ethylenic function, or the at least one monomer comprising at least one unsaturated ethylenic function, are selected from the group consisting of from acrylamide, acrylic acid, an acrylic acid oligomer, 2-acrylamido-2-methylpropanesulfonic acid (ATBS) and/or salts thereof, N-vinylformamide (NVF), N-vinylpyrrolidone (NVP), dimethyldiallylammonium chloride (DADMAC), quaternized dimethylaminoethyl acrylate (ADAME), and quaternized dimethylaminoethyl methacrylate (MADAME).

19. The hybrid polymer according to claim 14, wherein the protein units, or the at least one protein, are plant and/or animal proteins.

20. The hybrid polymer according to claim 14, wherein the protein units, or the at least one protein, are a casein, a serum protein or wheat protein.

21. The hybrid polymer according to claim 14, wherein hybrid polymer has a weight-average molecular weight is between 20 000 and 20 000 000 daltons.

22. The hybrid polymer according to claim 14, wherein the hybrid polymer is in a form of an inverse emulsion.

23. A composition comprising the hybrid polymer according to claim 14, wherein the hybrid polymer is a flocculant, coagulant, binder, fixative, viscosity reducer, thickener, absorbent, friction reducer, dewatering agent, drainage aid, filler retention agent, dehydrating agent, conditioner, stabilizer, film-forming agent, sizing agent, superplasticizer, clay inhibitor or dispersant.

24. A method of applying the hybrid polymer according to claim 14 to a field of application, comprising adding the hybrid polymer to a composition used in the field of application, wherein the field of application is selected from the group consisting of recovering hydrocarbons, drilling and cementing of wells, stimulating hydrocarbon wells, treating of water, treating fermentation musts in treating sludges, manufacturing paper, construction, treating wood, treating hydraulic compositions, mining, formulating cosmetic products, formulating of detergents, manufacturing textiles, manufacturing battery components, geothermal applications, manufacturing diapers, and agriculture applications.

25. An aqueous composition comprising at least one hybrid polymer according to claim 14, said composition comprising between 0.001% and 5% by weight of said hybrid polymer (HP).

26. A method of utilizing the composition according to claim 25 to a field of application, comprising applying the composition in the field of application, wherein the field of application is selected from the group consisting of recovering hydrocarbons, drilling and cementing of wells, stimulating hydrocarbon wells, treating of water, treating fermentation musts in treating sludges, manufacturing paper, construction, treating wood, treating hydraulic compositions, mining, formulating cosmetic products, formulating of detergents, manufacturing textiles, manufacturing battery components, geothermal applications, manufacturing diapers, and agriculture applications.
